(19) United States
(12) Patent Application Publication
(10) Pub. No.: US 2026/0009725 A1
(43) Pub. Date: Jan. 8, 2026

(54) SYSTEMS AND METHODS FOR DETERMINING CARBON DIOXIDE CONCENTRATIONS USING PEAK RATIO-BASED OPTICAL SPECTROMETRIC MEASUREMENTS

(71) Applicant: Schlumberger Technology Corporation, Sugar Land, TX (US)

(72) Inventors: Kai HSU, Sugar Land, TX (US); Yoko MORIKAMI, Sagamihara (JP); Albert Ballard ANDREWS, Cambridge, MA (US); Evgeniya DEGER, Sugar Land, TX (US); Thomas PFEIFFER, Katy, TX (US); Hua CHEN, Sugar Land, TX (US)

(21) Appl. No.: 19/136,310

(22) PCT Filed: Dec. 13, 2023
(86) PCT No.: PCT/US2023/083796

§ 371 Date: Jun. 6, 2025

Related U.S. Application Data
(60) Provisional application No. 63/387,175, filed on Dec. 13, 2022.

(51) Int. Cl. G01N 21/31; E21B 47/002; G01N 33/00; G01N 33/24

(52) U.S. Cl. CPC G01N 21/3151; E21B 47/002; G01N 33/0022; G01N 33/004; G01N 33/0062; G01N 33/241

(57) ABSTRACT

The disclosed techniques relate to techniques for determining a CO₂ concentration based on a ratio of one or more peaks in carbon dioxide (CO₂) optical spectrometer measurement data. For example, the techniques include receiving CO₂ measurement data corresponding to a region within a geological formation; identifying a peak corresponding to CO₂ based on the CO₂ measurement data; determining a ratio between the peak and a hydrocarbon reference measurement; selecting a model for determining a CO₂ concentration based on the ratio; determining the CO₂ concentration using the selected model; and generating a downhole operation output based on the determined CO₂ concentration.

### CROSS-REFERENCE TO RELATED APPLICATIONS

The present application is an International Application that claims priority to U.S. Provisional Patent Application No. 63/387,175 that was filed on Dec. 13, 2022, which is herein incorporated by reference in its entirety.

### FIELD

The present disclosure relates generally to downhole tools. More specifically, the present disclosure relates to techniques to improve the accuracy of determining an amount or concentration of a fluid, such as CO₂.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

The oil and gas industry includes a number of sub-industries, such as exploration, drilling, logging, extraction, transportation, refinement, retail, and so forth. During exploration and drilling, wellbores may be drilled into the ground for reasons that may include discovery, observation, and/or extraction of resources. These resources may include oil, gas, water, or any other combination of elements within the ground.

Wellbores or boreholes may be drilled to, for example, locate and produce hydrocarbons. During a well development operation, it may be desirable to evaluate and/or measure properties of encountered formations, formation fluids and/or formation gasses. For example, crude oil wells may include components such as carbon dioxide (CO₂) dissolved within the formation, which may affect certain properties of the formation fluid. While it may be advantageous to detect such fluids, and thus determine the proper, it may be difficult to accurately determine a concentration of such fluids (e.g., CO₂) using optical spectrometric data.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In some embodiments, a method is disclosed. The method may include receiving, via one or more processors, carbon dioxide (CO₂) optical spectrometer measurement data corresponding to a region within a geological formation. Further, the method may include identifying, via the one or processors, a peak corresponding to CO₂ based on the CO₂ measurement data, determining, via the one or more processors, a ratio between the peak and a hydrocarbon reference measurement, selecting, via the one or more processors, a model from a plurality of stored models for determining a CO₂ concentration based on the ratio, and determining, via the one or more processors, the CO₂ concentration using the selected model. Furthermore, the method may include generating, via the one or more processors, a downhole operation output based on the determined CO₂ concentration.

In other embodiments, a method is disclosed that may include receiving, via one or more processors, carbon dioxide (CO₂) optical spectrometer measurement data corresponding to a region within a geological formation. Further, the method may include identifying, via the one or processors, a peak corresponding to CO₂ based on the CO₂ measurement data, determining, via the one or more processors, a plurality of ratios between the peak corresponding to CO₂ and a plurality of hydrocarbon peak measurements, comparing, via the one or more processors, a ratio of the plurality of ratios to a threshold ratio, selecting, via the one or more processors, a model from a plurality of stored models for determining a CO₂ concentration based on the comparison between the ratio and the threshold ratio, and determining, via the one or more processors, the CO₂ concentration using the selected model. Furthermore, the method may include generating, via the one or more processors, a downhole operation output based on the determined CO₂ concentration.

Additionally, in one or more embodiments, a non-transitory computer-readable medium is disclosed. The non-transitory computer-readable medium may include computer-executable instructions that, when executed by a processor, are configured to cause the processor to perform one or more methods as disclosed above and herein.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings in which:

FIG. 1A is schematic diagram of downhole drilling system include an optical spectrometer system, in accordance with aspects of the present disclosure;

FIG. 1B is a schematic diagram of downhole equipment having various testing modules used to determine one or more characteristics of the subsurface formation, in accordance with an embodiment of the present techniques;

FIG. 2 is a graph depicting measured carbon dioxide (CO₂) weight percent (wt %) versus predicted CO₂ weight percent (wt %) using a model;

FIG. 3 is a graph depicting CO₂ peaks versus CO₂ wt %;

FIG. 4 is a graph depicting CO₂ peak ratios normalized by the optical density (OD) at 1690 nanometers (nm) versus CO₂ wt %, in accordance with aspects of the present disclosure;

FIG. 5 is a graph depicting CO₂ peak ratios normalized by the optical density (OD) at 1725 nanometers (nm) versus CO₂ wt %, in accordance with aspects of the present disclosure;

FIG. 6 is a graph depicting CO₂ peak ratios normalized by the optical density (OD) at 1760 nanometers (nm) versus CO₂ wt %, in accordance with aspects of the present disclosure;

FIG. 7 is a graph depicting CO₂ peak ratios normalized by the optical density (OD) at 1800 nanometers (nm) versus CO₂ wt %, in accordance with aspects of the present disclosure;

FIG. 8 is a graph depicting CO₂ peak ratios at 1650 nm, 1671 nm, 1690 nm, 1725 nm, 1760 nm, and 1800 nm versus CO₂ wt %, in accordance with aspects of the present disclosure;

FIG. 9 is a graph depicting measured CO₂ wt % versus predicted CO₂ wt % based on determining whether CO₂ peak ratios exceed a threshold, in accordance with aspects of the present disclosure;

FIG. 10 is a flow diagram of a method for generating a downhole operation output based on a determined peak ratio using optical spectrometric measurement data, in accordance with aspects of the present disclosure;

FIG. 11 is a graph depicting optical spectrometric data (OD) versus wavelength, in accordance with aspects of the present disclosure;

FIG. 12 shows a first example spectral data and estimated CO₂ wt %, in accordance with aspects of the present disclosure;

FIG. 13 shows additional spectral data in accordance with aspects of the present disclosure; and

FIG. 14 shows a second example spectral data and estimated CO₂ wt %, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and enterprise-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

As used herein, the terms “connect,” “connection,” “connected,” “in connection with,” and “connecting” are used to mean “in direct connection with” or “in connection with via one or more elements”; and the term “set” is used to mean “one element” or “more than one element.” Further, the terms “couple,” “coupling,” “coupled,” “coupled together,” and “coupled with” are used to mean “directly coupled together” or “coupled together via one or more elements.” As used herein, the terms “up” and “down,” “uphole” and “downhole”, “upper” and “lower,” “top” and “bottom,” and other like terms indicating relative positions to a given point or element are utilized to more clearly describe some elements. Commonly, these terms relate to a reference point as the surface from which drilling operations are initiated as being the top (e.g., uphole or upper) point and the total depth along the drilling axis being the lowest (e.g., downhole or lower) point, whether the well (e.g., wellbore, borehole) is vertical, horizontal or slanted relative to the surface.

As used herein, the term “computing system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the term “medium” refers to one or more non-transitory, computer-readable physical media that together store the contents described as being stored thereon. Embodiments may include non-volatile secondary storage, read-only memory (ROM), and/or random-access memory (RAM). As used herein, the term “application” refers to one or more computing modules, programs, processes, workloads, threads and/or a set of computing instructions executed by a computing system. Example embodiments of an application include software modules, software objects, software instances and/or other types of executable code.

In addition, as used herein, the terms “real time”, “real-time”, or “substantially real time” may be used interchangeably and are intended to describe operations (e.g., computing operations) that are performed without any human-perceivable interruption between operations. For example, as used herein, data relating to the systems described herein may be collected, transmitted, and/or used in control computations in “substantially real time” such that data readings, data transfers, and/or data processing steps occur once every second, once every 0.1 second, once every 0.01 second, or even more frequently, during operations of the systems (e.g., while the systems are operating). In addition, as used herein, the terms “continuous”, “continuously”, or “continually” are intended to describe operations that are performed without any significant interruption. For example, as used herein, control commands may be transmitted to certain equipment every five minutes, every minute, every 30 seconds, every 15 seconds, every 10 seconds, every 5 seconds, or even more often, such that operating parameters of the equipment may be adjusted without any significant interruption to the closed-loop control of the equipment.

In addition, as used herein, the terms “automatic”, “automated”, “autonomous”, and so forth, are intended to describe operations that are performed are caused to be performed, for example, by a computing system (i.e., solely by the computing system, without human intervention). Indeed, it will be appreciated that the data processing systems and control systems described herein may be configured to perform any and all of the data processing and control functions described herein automatically.

As mentioned above, it may be useful to determine a concentration or amount of CO₂ within a well to aid in oil and gas operations, such as recovery. For example, the concentration or amount of CO₂ may impact decisions of field development. That is, if any or a suitable amount of CO₂ is present within the well, it may be advantageous to modify a development plan to account for capturing the CO₂. In general, CO₂ may be detected using certain spectroscopy techniques, such as infrared spectroscopy, where a sensor may detect a presence or concentration of CO₂ by detecting one or more molecular vibrations in near infrared. For example, a formation tester may include one or more optical spectrometers (e.g., downhole spectrometers) to obtain, acquire, or measure transmittance data (e.g., optical density (OD) versus wavelength). In general, the optical spectrometers may include a light source, a flow line having optical windows through which the light is transmitted, and one or more detectors. As described in more detail below, the one or more detectors may include multi-channel detectors that measure the intensity of the transmitted light (i.e., through the optical windows) at one or more predetermined wavelengths.

More specifically, certain downhole spectrometers may allocate one or more wavelength channels around the CO₂ absorption peaks that correspond to CO₂ molecular vibrations for detecting and estimating concentration of the CO₂. In certain conventional techniques, three wavelength channels at 1980 nm, 2010 nm and 2040 nm are utilized to detect CO₂. Further, a processor may utilize certain CO₂ algorithms based on downhole spectral data. Certain algorithms may be based on the same principle of simultaneously estimating all hydrocarbon components (i.e. C₁, C₂, C₃, C₄, C₅ and C₆₊) and CO₂ using a mapping approach. Additional algorithms may be based on a different logic which sequentially estimates C₁, C₂, C₃₋₅, C₆₊ and at the end, the CO₂ component is estimated based on the CO₂ absorption peaks around 2010 nm and all previously estimated hydrocarbon components. However, it is presently recognized that conventional techniques may not accurately determine CO₂ concentrations for certain ranges, particularly when the CO₂ concentration is relatively high (e.g., 50 wt % or greater, 55 wt % or greater, 60 wt % or greater, 65 wt % or greater, and so on).

Accordingly, the present disclosure is directed to a CO₂ peak ratio-based analysis technique to increase the accuracy of determining a CO₂ amount or concentration for both relatively low CO₂ concentrations (e.g., 35 wt % or less, 40 wt % or less, 45 wt % or less, 50 wt % or less, 55 wt % or less, 60 wt % or less, and so on) and relatively high CO₂ concentrations. The disclosed CO₂ ratio base analysis technique generally includes receiving CO₂ measurement data, such as spectra acquired by a downhole spectrometer, and determining a ratio between the CO₂ measurement data (e.g., a CO₂ peak) versus a reference value (e.g., hydrocarbon reference measurement). In general, the reference value may be an optical density or absorption coefficient at a particular wavelength, wavelength range, frequency, or frequency range corresponding to one or more hydrocarbon reference measurements or peaks. Further, the CO₂ peak ratio-based analysis techniques include comparing the ratio to a threshold ratio. If the ratio exceeds the threshold ratio, a processor may retrieve a first model to determine a concentration of CO₂ from the CO₂ peak-ratio measurement data. However, if the ratio is below the threshold ratio, the processor may retrieve a second model (e.g. different from the first model) to determine the concentration of CO₂ from the CO₂ peak-ratio measurement. In general, it is recognized that certain models may be more efficient and/or more accurate at determining a CO₂ concentration or amount when the CO₂ wt % is relatively high. Similarly, certain other models may be more efficient and/or more accurate at determining the CO₂ concentration when the CO₂ wt % is relatively low. However, determining which model to use to determine the CO₂ concentration may be difficult as a user may be unable to estimate the CO₂ concentration before acquiring the CO₂ measurement data. In this way, the disclosed CO₂ ratio-based analysis techniques may provide more accurate CO₂ concentrations to better inform certain oil and gas related decisions. Moreover, the disclosed techniques may reduce the allocation of computational resources by, for example, determining a model from multiple stored models that provides more accurate fluid property data (e.g., a concentration of CO₂).

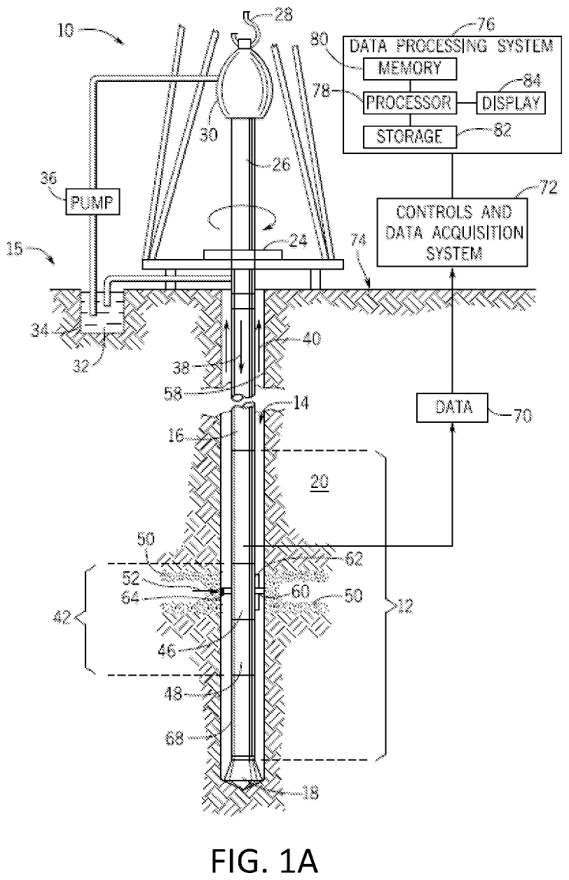

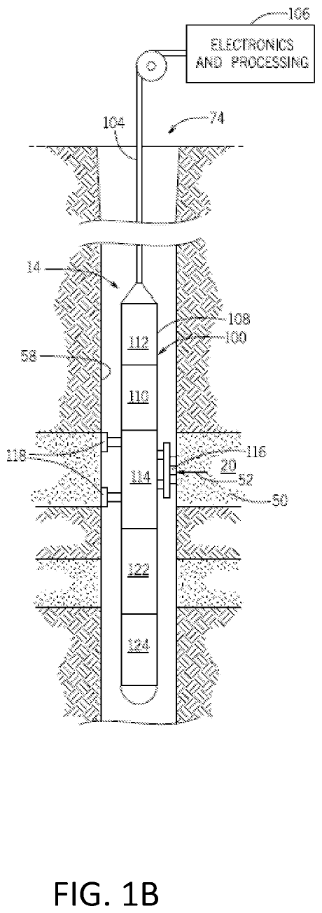

With the foregoing in mind, FIG. 1A depicts an example of wellsite systems that may employ the techniques described herein. FIG. 1A depicts a rig 10 with a downhole tool 12 suspended therefrom and into a wellbore 14 of a reservoir via a toolstring 16. The drill string 16 is rotated by a rotary table 24, energized by means not shown, which engages a kelly 26 at the upper end of the drill string 16. The drill string 16 is suspended from a hook 28, attached to a traveling block (also not shown), through the kelly 26 and a swivel 30 (e.g., rotary swivel) that permits rotation of the drill string 16 relative to the hook 28. The rig 10 is depicted as a land-based platform and derrick assembly used to form the wellbore 14 by rotary drilling.

While the depicted embodiment relates to a downhole tool 12 disposed in a wellbore 14, it should be understood that, at least in some instances, the disclosure techniques may be used in a logging-while drilling (LWD) tool. In such an embodiment, the formation fluid or drilling mud 32 (e.g., oil base mud (OBM) or water-based mud (WBM)) may be stored in a pit 34 formed at the well site. A pump 36 delivers the reservoir fluid 52 to the interior of the drill string 16 via a port in the swivel 30, inducing the drilling mud 32 to flow downwardly through the drill string 16 as indicated by a directional arrow 38. The formation fluid exits the drill string 16 via ports of the downhole tool 12, and then circulates upwardly through the region between the outside of the drill string 16 and the wall of the wellbore 14, called the annulus, as indicated by directional arrows 40. The drilling mud 32 lubricates a drill bit and carries formation cuttings up to the surface as it is returned to the pit 34 for recirculation.

In certain embodiments, the downhole tool 12 includes a downhole analysis system. For example, the downhole tool 12 may include a sampling system 42 including a fluid communication module 46 and a sampling module 48. The modules may be housed in a drill collar for performing various formation evaluation functions, such as pressure testing and fluid sampling, among others. As shown in FIG. 1, the fluid communication module 46 is positioned adjacent the sampling module 48; however, the position of the fluid communication module 46, as well as other modules, may vary in other embodiments. Additional devices, such as pumps, gauges, sensor, monitors or other devices usable in downhole sampling and/or testing also may be provided. The additional devices may be incorporated into the fluid communication module 46, the sample module 48, or disposed within separate modules included within the sampling system 42.

In some embodiments, the downhole tool 12 may be a formation testing downhole tool. For example, the downhole tool 12 may evaluate fluid properties of reservoir fluid 50. Accordingly, the sampling system 42 may include sensors that may measure fluid properties such as gas-to-oil ratio (GOR), mass density, optical density (OD), composition of C₁, C₂, C₃, C₄, C₅, and C₆₊, formation volume factor, viscosity, resistivity, fluorescence, American Petroleum Institute (API) gravity, and combinations thereof of the reservoir fluid 50. In certain embodiments, the fluid communication module 46 includes a probe which may be positioned inside borehole. In addition, in certain embodiments, the probe includes one or more inlets for receiving the reservoir fluid 52 and one or more flowlines (not shown) extending into the downhole tool 12 for passing fluids (e.g., the reservoir fluid 50) through the tool. In certain embodiments, the probe may include a single inlet designed to direct the reservoir fluid 50 into a flowline within the downhole tool 12. Further, in other embodiments, the probe may include multiple inlets that may, for example, be used for focused sampling. In these embodiments, the probe may be connected to a sampling flowline, as well as to guard flowlines. In certain embodiments, the probe may be movable between extended and retracted positions for selectively engaging the wellbore wall 58 of the wellbore 14 and acquiring fluid samples from the geological formation 20. One or more setting accessories, standoffs, or rollers 64 may be provided to assist in positioning the fluid communication device against the wellbore wall 58.

In certain embodiments, the downhole tool 12 includes a spectral analysis module 68. The spectral analysis module 68 includes a radiation source that emits radiation (e.g., gamma rays) into the geological formation 20 to determine formation properties such as, for example, lithology, density, formation geometry, reservoir boundaries, among others. The gamma rays interact with the formation 20, which may attenuate the gamma rays. Sensors within the spectral analysis module 68 may detect the scattered gamma rays and determine the geological characteristics of the geological formation 20 based at least in part on the attenuated gamma rays. In some embodiments, the downhole tool 12 may include one or both of the spectral analysis module 68 and the fluid analyzer module 47.

FIG. 1B depicts an example of a wireline downhole tool 100 that may employ the systems and techniques described herein to determine a CO₂ concentration of the reservoir fluid 50. The wireline downhole tool 100 is suspended in the wellbore 14 from the lower end of a multi-conductor cable 104 that is spooled on a winch at the surface 74. Similar to the downhole acquisition tool 12, the wireline downhole tool 100 may be conveyed on wired drill pipe, a combination of wired drill pipe and wireline, or other suitable types of conveyance. The cable 104 is communicatively coupled to an electronics and processing system 106. The wireline downhole tool 100 includes an elongated body 108 that houses modules 110, 112, 114, 122, and 124 that provide various functionalities including imaging, fluid sampling, fluid testing, operational control, and communication, among others. For example, the modules 110 and 112 may provide additional functionality such as fluid analysis, resistivity measurements, operational control, communications, coring, and/or imaging, among others.

As shown in FIG. 1B, the module 114 is a fluid communication module 114 that has a selectively extendable probe 116 and backup pistons 118 that are arranged on opposite sides of the elongated body 108. The extendable probe 116 is configured to selectively seal off or isolate selected portions of the wall 58 of the wellbore 14 to fluidly couple to the adjacent geological formation 20 and/or to draw fluid samples from the geological formation 20. The probe 116 may include a single inlet or multiple inlets designed for guarded or focused sampling. The reservoir fluid 50 may be expelled to the wellbore through a port in the body 108 or the formation fluid 50 may be sent to one or more modules 122 and 124. The modules 122 and 124 may include sample chambers that store the reservoir fluid 50. In the illustrated example, the electronics and processing system 106 and/or a downhole control system are configured to control the extendable probe assembly 116 and/or the drawing of a fluid sample from the formation 20 to enable analysis of the fluid properties of the reservoir fluid 50, as discussed above. In some embodiments, the wireline downhole tool 100 may include one or more light sources and/or light detectors disposed along a fluid conduit of the wireline downhole tool 100 to facilitate acquiring optical spectrometer data of the reservoir fluid 50.

In certain embodiments, the sensors within the downhole tool 12 may collect and transmit data 70 associated with the characteristics of the geological formation 20 and/or the fluid properties and the composition of the reservoir fluid 50 to a control and data acquisition system 72 at surface 74, where the data 70 may be stored and processed in a data processing system 76 of the control and data acquisition system 72.

The data processing system 76 may include a processor 78, memory 80, storage 82, and/or display 84. The memory 80 may include one or more tangible, non-transitory, machine readable media collectively storing one or more sets of instructions for operating the downhole tool 12, determining formation characteristics (e.g., geometry, connectivity, minimum horizontal stress, etc.) calculating and estimating fluid properties of the reservoir fluid 50, modeling the fluid behaviors using, e.g., equation of state models (EOS). The memory 80 may store reservoir modeling systems (e.g., geological process models, petroleum systems models, reservoir dynamics models, etc.), mixing rules and models associated with compositional characteristics of the reservoir fluid 50, equation of state (EOS) models for equilibrium and dynamic fluid behaviors (e.g., biodegradation, gas/condensate charge into oil, CO₂ charge into oil, fault block migration/subsidence, convective currents, among others not related to methane hydrate), and any other information that may be used to determine geological and fluid characteristics of the geological formation 20 and reservoir fluid 52, respectively. In certain embodiments, the data processing system 76 may apply filters to remove noise from the data 70.

To process the data 70, the processor 78 may execute instructions stored in the memory 80 and/or storage 82. For example, the instructions may cause the processor to compare the data 70 (e.g., from the logging while drilling and/or downhole analysis) with known reservoir properties estimated using the reservoir modeling systems, use the data 70 as inputs for the reservoir modeling systems, and identify geological and reservoir fluid parameters that may be used for exploration and production of the reservoir. As such, the memory 80 and/or storage 82 of the data processing system 76 may be any suitable article of manufacture that can store the instructions. By way of example, the memory 80 and/or the storage 82 may be ROM memory, random-access memory (RAM), flash memory, an optical storage medium, or a hard disk drive. The display 84 may be any suitable electronic display that can display information (e.g., logs, tables, cross-plots, reservoir maps, etc.) relating to properties of the well/reservoir as measured by the downhole tool 12. It should be appreciated that, although the data processing system 76 is shown by way of example as being located at the surface 74, the data processing system 76 may be located in the downhole tool 12. In such embodiments, some of the data 70 may be processed and stored downhole (e.g., within the wellbore 14), while some of the data 70 may be sent to the surface 74 (e.g., in real time). In certain embodiments, the data processing system 76 may use information obtained from petroleum system modeling operations, ad hoc assertions from the operator, empirical historical data (e.g., case study reservoir data) in combination with or lieu of the data 70 to determine certain parameters of the reservoir 15.

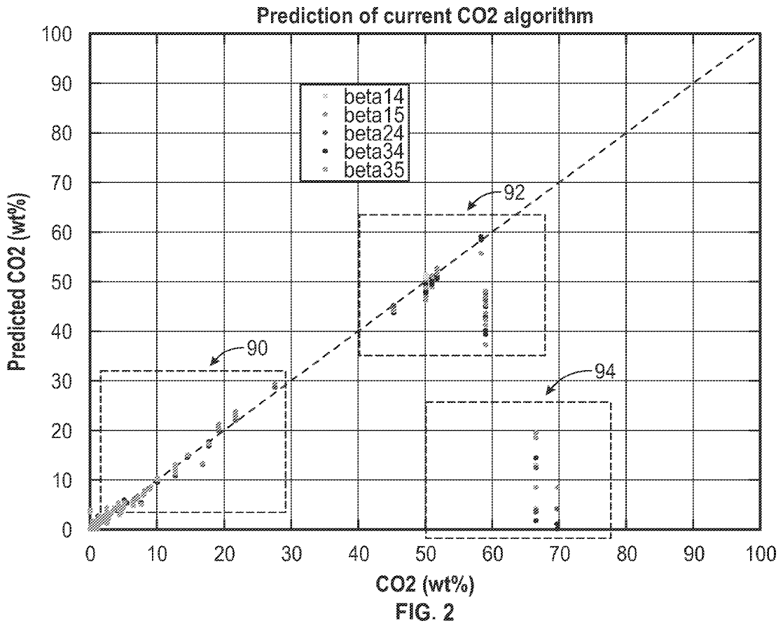

As described herein, the disclosed techniques relate to determining the CO₂ concentration using downhole spectrometer data. To illustrate this, FIG. 2 is a graph having a horizontal or x-axis corresponding to a measured CO₂ wt % and a vertical or y-axis corresponding to a predicted CO₂ wt %. In general, when utilizing a model to determine a CO₂ concentration or amount, there may be an unknown relationship (i.e., or model) between the spectral data and CO₂ concentration that is solved by a training (i.e., calibrating) procedure, such as Partial Least Squares (PLS). However, the training data in the database may be limited to the data with relatively low CO₂ concentrations (e.g., less than 30%) corresponding to the region 130 and relatively mid-ranged CO₂ concentrations (e.g., less than 60%) corresponding to the region 132, and therefore, the built model may only be valid in for relatively low and relatively mid-ranged CO₂ concentrations.

Provided herein is an example technique for determining a fluid properties, such as a CO₂ concentration, using spectral data or acquired optical property measurements (e.g., a transmittance measurement) at multiple wavelengths. In general, the multiple wavelengths may include different discrete wavelengths, a range of wavelengths, or a combination thereof that corresponds to one or more chemical species or molecules. For example, to detect certain carbon species such as methane (C₁), ethane (C₂), propane (C₃), butane (C₄), pentane (C₅), hexane or alkanes with more than six carbons (C₆₊), and CO₂, or any combination thereof, an optical spectrometer may acquire spectral data (e.g., optical property data) at multiple wavelengths between 1500 and 2300 nm. In general, each of the carbon species may have one or more peaks corresponding to vibrational modes of the carbon species. One or more of the carbon may overlap within a particular wavelength range. For example, C₁-C₅, C₆₊, and CO₂ may have peaks that overlap between 1500 and 2300 nm. Based on the Beer Lambert's Law, the measured optical density can be written as:

$$OD ( λ ) = {\sum }_{i = 1}^{7} {δ}_{i} ( λ ) {ρ}_{i} l = {\sum }_{i = 1}^{7} {u}_{i} ( λ ) {ρ}_{i} , i ∈ \{ {C}_{1} , {C}_{2} , {C}_{3} , {C}_{4} , {C}_{5} , {C}_{6 +} , {CO}_{2} \} \qquad ( 1 )$$

where δᵢ(λ) is the absorption coefficient of i^{th} component at wavelength λ, ρᵢ is the concentration of i^{th} component and l is the optical path length, and uᵢ(λ)=δᵢ (λ) l. For downhole spectrometer, the CO2 absorption only occurs at the wavelength channel of 2010 nm. The absorption of other hydrocarbon components, however, also present at this channel and are superimposed on the absorption of CO2.

The peak of CO2 (i.e. p) may be defined at 2010 nm as the OD magnitude after removing the baseline (see FIG. 1) which is defined by the two neighboring channels (i.e. 1985 nm and 2040 nm), i.e. let λ₁=2010 nm, λ_{b}=1985 nm and λ₃=2040 nm:

$$p = OD ( {λ}_{1} ) - \frac{{λ}_{3} - {λ}_{1}}{{λ}_{3} - {λ}_{2}} OD ( {λ}_{2} ) - \frac{{λ}_{1} - {λ}_{2}}{{λ}_{3} - {λ}_{2}} OD ( {λ}_{3} ) \qquad ( 2 a )$$

$$= OD ( {λ}_{1} ) - \frac{6}{11} OD ( {λ}_{2} ) - \frac{5}{11} OD ( {λ}_{3} ) \qquad ( 2 b )$$

$$p ( {λ}_{1} , {λ}_{2} , {λ}_{3} ) = {\sum }_{i = 1}^{6} ( {u}_{i} ( {λ}_{1} ) - \frac{6}{11} {u}_{i} ( {λ}_{2} ) - \frac{5}{11} {u}_{i} ( {λ}_{3} ) ) {ρ}_{i} + {u}_{CO 2} ( {λ}_{1} ) {ρ}_{CO 2} \qquad ( 3 )$$

$$where :$$

$$OD ( {λ}_{1} ) = {\sum }_{i = 1}^{6} {u}_{i} ( {λ}_{1} ) {ρ}_{i} + {u}_{CO 2} ( {λ}_{1} ) {ρ}_{CO 2} \qquad ( 4 )$$

$$OD ( {λ}_{2} ) = {\sum }_{i = 1}^{6} {u}_{i} ( {λ}_{2} ) {ρ}_{i} \qquad ( 5 )$$

$$OD ( {λ}_{3} ) = {\sum }_{i = 1}^{6} {u}_{i} ( {λ}_{3} ) {ρ}_{i} \qquad ( 6 )$$

At least in some instances, it may be advantageous to remove the OD dependence on temperature and pressure. To do so, the peak of CO₂ (i.e., p) may be normalized to a hydrocarbon absorption peak in the hydrocarbon absorption region (i.e. 1600 nm-1800 nm). For example, let λ₄=1690 nm and λ_{b}=1600 nm, the magnitude of hydrocarbon peak at λ₄ may be defined as:

$$h ( {λ}_{4} , {λ}_{b} ) = OD ( {λ}_{4} ) - OD ( {λ}_{b} ) = {\sum }_{i = 1}^{6} ( {u}_{i} ( {λ}_{4} ) - {u}_{i} ( {λ}_{b} ) ) {ρ}_{i} \qquad ( 7 )$$

The normalization may be performed by taking the ratio of p and h, i.e.:

$$r ( {λ}_{1} , {λ}_{2} , {λ}_{3} , {λ}_{4} , {λ}_{b} ) = \frac{p ( {λ}_{1} . {λ}_{2} , {λ}_{3} )}{h ( {λ}_{4} , {λ}_{b} )} \qquad ( 8 )$$

It should be noted that for computing the peak ratio in equation (Eqn.) 8, one can choose λ₄ to be any other hydrocarbon absorption channel (e.g. 1650 nm, 1671 nm, 1725 nm, 1760 nm and 1800 nm) in the hydrocarbon region.

Based on Eqn. 8, the peak ratio can be written as:

$$r = \frac{{\sum }_{i = 1}^{i = 6} {α}_{i} {ρ}_{i} + {ρ}_{C O 2}}{{\sum }_{i = 1}^{i = 6} {β}_{i} {ρ}_{i}} \qquad ( 9 )$$

$$Where :$$

$${α}_{i} ( {λ}_{1} , {λ}_{2} , {λ}_{3} ) = \frac{{u}_{i} ( {λ}_{1} ) - \frac{6}{11} {u}_{i} ( {λ}_{2} ) - \frac{5}{11} {u}_{i} ( {λ}_{3} )}{{u}_{CO 2} ( {λ}_{1} )} \qquad ( 10 )$$

$${β}_{i} ( {λ}_{1} , {λ}_{4} , {λ}_{b} ) = \frac{{u}_{i} ( {λ}_{4} ) - {u}_{i} ( {λ}_{b} )}{{u}_{CO 2} ( {λ}_{1} )} \qquad ( 11 )$$

Furthermore, the peak ratio can be written in terms of a weighted fraction of each component (i.e. wᵢ) by dividing the denominator and nominator of Eqn. 9 by the fluid density ρ:

$$r = \frac{{\sum }_{i = 1}^{i = 6} {α}_{i} {w}_{i} + {w}_{C O 2}}{{\sum }_{i = 1}^{i = 6} {β}_{i} {w}_{i}} \qquad ( 12 )$$

$$ρ = {\sum }_{i = 1}^{i = 7} {ρ}_{i} , i ⊆ \{ {C}_{1} , {C}_{2} , {C}_{3} , {C}_{4} , {C}_{5} , {C}_{6 +} , {CO}_{2} \} \qquad ( 13 )$$

Therefore, from Eqn. 12, one may obtain:

$${w}_{C O 2} = r {\sum }_{i = 1}^{i = 6} {β}_{i} {w}_{i} + {\sum }_{i = 1}^{i = 6} {α}_{i} {w}_{i} \qquad ( 14 )$$

A first technique for determining CO₂ concentration may be based on Eqn 14, which states—the weight fraction of CO2 (left-hand-side of Eqn. (14)) can be estimated using the CO₂ peak ratio (i.e. r) and the hydrocarbon weight fractions (i.e. wᵢ). The unknowns α₁ and β₁ in Eqn. 14 may be obtained by calibrating against the reference spectra data, which contain live fluids with various amount of CO2 concentrations.

The reference spectra data may include optical spectra (e.g., OD versus wavelength) of a wide variety of fluid samples measured at various temperatures (e.g., up to 175° C.) and pressures (up to 25000 psi), and their corresponding compositions (i.e. weight fractions derived from gas chromatography analysis) and PVT properties. Based on their fluid properties, the spectral data may be divided into different categories, such as gas, gas condensate and oil.

Calibration (i.e., training) may include computing the CO2 peak ratios from the database spectra and using the corresponding hydrocarbon weight fractions and CO₂ weight fractions in the database to estimate the unknowns α₁ and β₁ based on Eqn. 14. This may be done by suitable methods, such as a least-squares multivariate regression. After determining or estimating α₁ and β₁ via the training procedure, they may be used with the peak ratio and the hydrocarbon weight fractions (i.e. wᵢ) to predict the CO2 weight fraction based on Eqn. 14.

To summarize the above technique, a CO₂ weight fraction, amount, or concentration may be determined using a suitable processor by receiving optical spectra including optical density versus wavelength at multiple wavelengths (i.e., corresponding to the different channels for different wavelengths as described herein). Then, the suitable processor may select a wavelength (e.g., λ₄) corresponding to a hydrocarbon peak. Further, the processor may determine a CO₂ peak ratio by using Eqn. 8, for example. Further still, the processor may determine the CO₂ weight fraction, amount, or concentration based on the CO₂ peak ratio, a weight fraction of other hydrocarbon components (e.g., wᵢ) and the determined parameters αᵢ and βᵢ.

The technique above may utilize weight fraction (i.e. wᵢ) of hydrocarbon components (i.e. C₁, C₂, C₃, C₄, C₅ and C₆₊) as input. However, at least in some instances, it may be advantageous to generate a model that does not utilize the weight fraction as input. In other words, the technique described below may estimate the CO2 weight fraction using the CO2 peak ratio measurements without the weight fraction.

It should be noted that there may be a general correlation trend between the CO₂ peak and CO₂ wt % but the trend may also be marked by an incoherent fluctuation, which may be at least partially caused by the OD dependence on temperature and pressure. As described herein, the temperature and pressure effect may be removed by normalizing the peak ration (e.g., peak of CO₂) to a hydrocarbon absorption peak in the hydrocarbon absorption region (i.e. 1600 nm-1800 nm). The trend between the CO₂ peak and the CO₂ wt % may not be linear. Instead, the trend may be a higher order trend, such as a quadratic trend with second-order dependence. In such instances, the following model is used:

$${w}_{C O 2} ∼ O ( p ) + O ( {p}^{2} ) \qquad ( 15 )$$

The unknown parameters in Eqn. 15 may be obtained by training (e.g., calibrating) using the reference spectra data as described above. In some embodiments, Partial Least Squares (PLS) may be used in the training for fluid composition. In other words, the PLS training based on Eqn. 15 may build a mapping matrix that may be utilized to determined a predicted CO₂ wt % using the peak ratio (i.e., p).

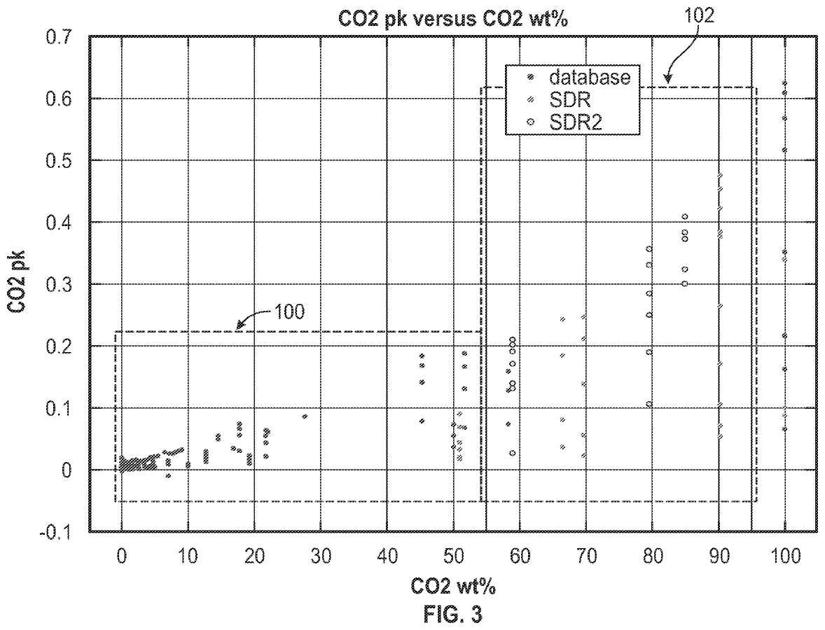

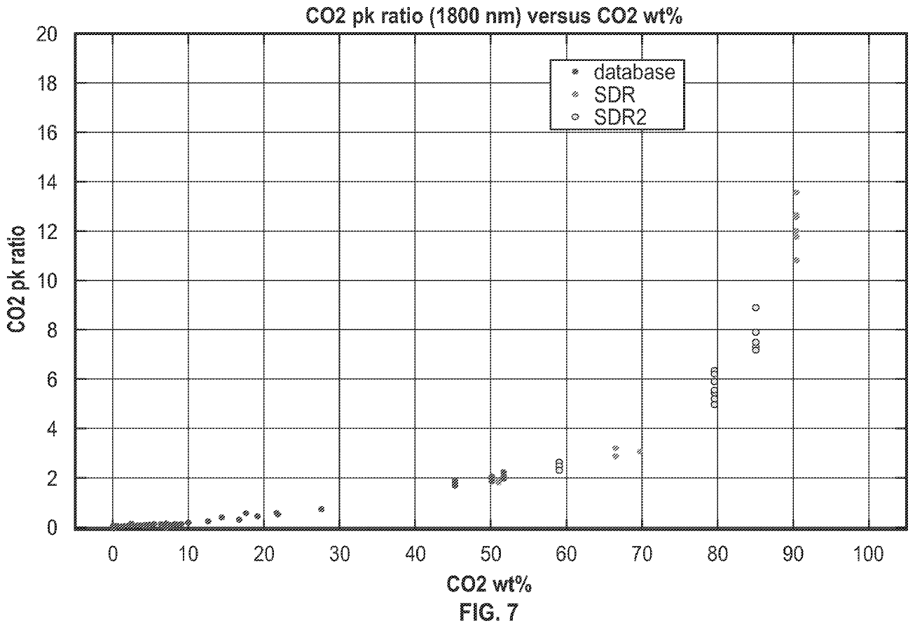

FIG. 7 shows the CO₂ prediction using the data in the spectral database. The peak ratios at 1690 nm and 1725 nm of spectra were jointly trained to obtain the mapping matrix. The mapping matrix is then used to predict the CO₂ wt % which are then compared with the database CO₂ wt %. The comparison is shown on the top subplot of FIG. 7. With respect to the diagonal line, the predicted CO₂ values show a good agreement with the corresponding database CO₂ values. The distribution of prediction errors is shown at the bottom of FIG. 3 with the mean absolute error (MAE) of about 0.49 wt %. Accordingly, this technique may be used for determining a CO₂ weight fraction, concentration, or amount using a mapping matrix and a computer CO₂ peak ratio.

It is presently recognized that it may be difficult to train a single model for both relatively low CO₂ concentration data (e.g., less than 50 wt %) and added relatively high CO₂ concentration data (e.g., greater than 50 wt %). To illustrate this, FIG. 2 shows a technique tested with relatively low CO₂ concentration data (e.g., less than 50 wt %) and added relatively high CO₂ concentration data (e.g., greater than 50 wt %) corresponding to the region 134. The graph illustrates results from five different models (i.e., beta14, beta15, beta24, beta34 and beta 35) to estimate the CO₂ concentration. The final CO₂ estimate is determined using the median value of the five estimates from the five models. As shown, the technique provides accurate (e.g., based on the diagonal trend line) predicted CO₂ wt % for relatively low CO₂ wt %. However, at the relatively mid-ranged CO₂ concentrations, there is a departure of the predicted CO₂ wt % from the measured CO₂ wt %, occurring generally within the region 92 between 50 wt % to 60 wt %. Beyond 60 wt % (i.e., within region 134), the predicted estimates even drop to negative values. Accordingly, utilizing this technique may provide inaccurate CO₂ wt % measurements if the CO₂ wt % is relatively high or mid-ranged. As noted before, the deficiency of the model for high concentration CO₂ data was originated from lack of high concentration CO₂ training data and therefore the built model performs unexpectedly (e.g., fails) for the high concentration CO₂ data. At least in some instances, these models may be trained using techniques such as machine learning (ML), artificial intelligence (AI), or a combination thereof. Further, the models may be trained using reference spectral data indicating a concentration of CO₂ corresponding to a measured optical density.

FIG. 3 is a graph having a horizontal or x-axis corresponding to a measured CO₂ wt % and a vertical or y-axis corresponding to a CO₂ peak (e.g., as described above with respect to Eqns. 8 and/or 9). In general, the data depicted in the graph of FIG. 3 extend the original database to include spectral data of binary mixtures (i.e., methane and CO₂) gas which contain relatively high CO₂ concentrations in laboratory. The original database were acquired in PVT laboratory for training the model used to predict the concentration of CO₂ (i.e. wt %) in FIG. 2.

In particular, FIG. 3 shows the CO₂ peak versus the CO₂ wt % of a first dataset (e.g., stored in a database) and a second dataset and third dataset acquired, obtained, or measured in two separate periods of time. As shown, the additional data fill up a gap of original database (shown as “blue” dots) in region 142 corresponding to the concentration of CO2 approximately 55-95 wt %. At least a portion of region 140 is filled in by the second dataset and/or the third dataset. This gap in region 142 is in the relatively high CO₂ concentration region that were not included in the original database for training. As shown in the graph, there is a general correlation trend between the CO₂ peak and CO₂ wt % but the trend is also marked by some large fluctuations, which are caused by the OD dependence on temperature and pressure. As noted, before, the temperature and pressure effect can be largely removed by computing the CO₂ peak ratio.

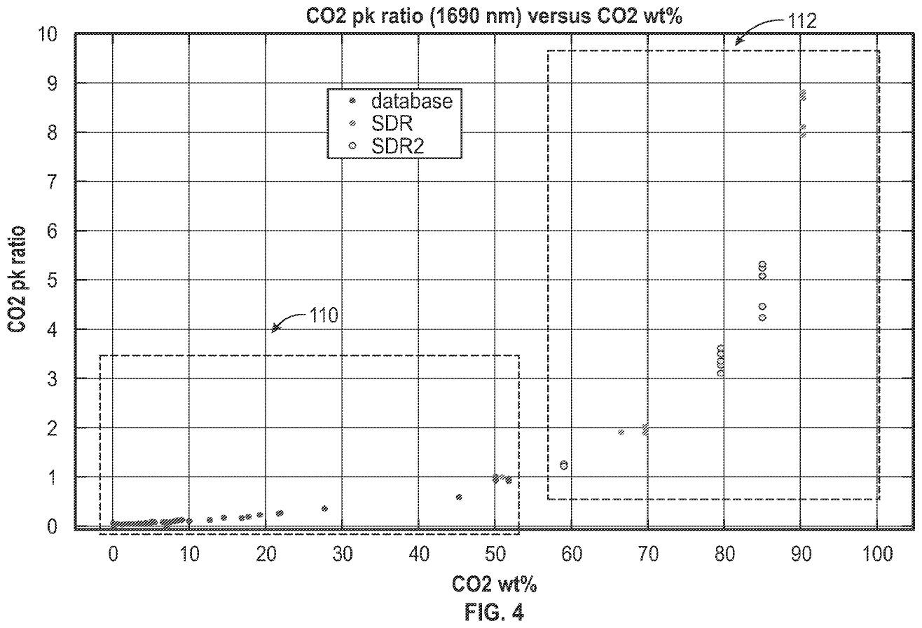

FIG. 4 is a graph having a horizontal or x-axis corresponding to a measured CO₂ wt % and a vertical or y-axis corresponding to a determined CO₂ peak ratio. In general, the peak ratio is a ratio of the identified peak of CO₂ measurement data relative to the reference channel data. In this case, the reference channel data is the optical density (OD) value at 1690 nm for the measurement data. However it should be appreciated that other reference data (i.e., different OD values at different wavelengths or wavelength ranges, such as an average or median of multiple wavelengths) may be used.

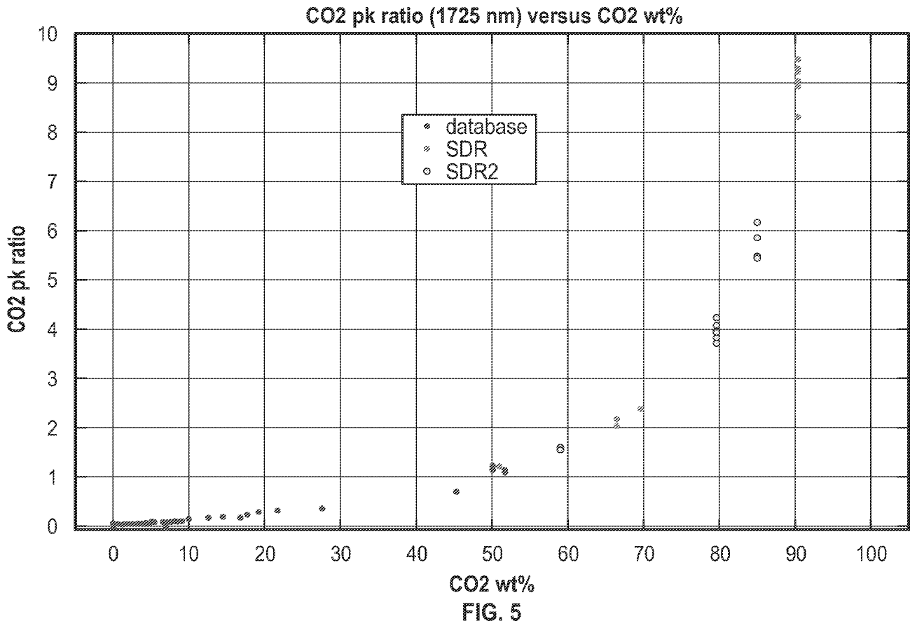

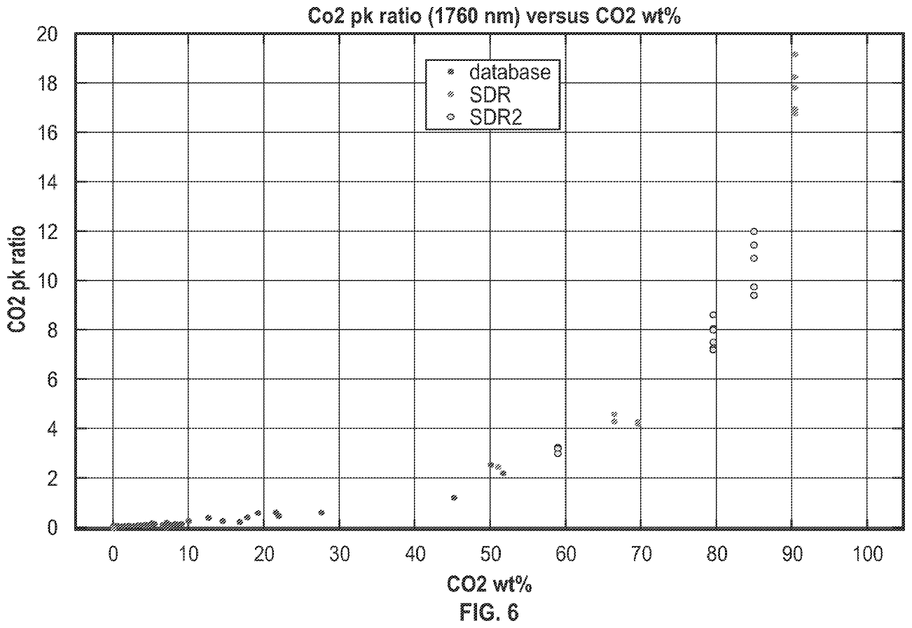

The graph shows the peak ratios at 1690 nm (i.e., the CO₂ peaks normalized by the OD value at 1690 nm) versus the CO₂ wt %. In general, the peak ratios are correlated with the CO₂ wt % in this case. Furthermore, the peak ratios alleviate the effect of temperature and pressure on the measurements as noted in FIG. 3. That is, there is a trend between the peak ratios within the relatively low CO₂ wt % region 150 and the relatively high CO₂ wt % region 152. Similarly, the peak ratios at other wavelengths also show clear correlation trends, as shown in FIGS. 5-7. More specifically, FIG. 5 is a graph having a horizontal or x-axis corresponding to a measured CO₂ wt % and a vertical or y-axis corresponding to a determined CO₂ peak ratio using a measured intensity at 1725 nm. FIG. 6 is a graph having a horizontal or x-axis corresponding to a measured CO₂ wt % and a vertical or y-axis corresponding to a determined CO₂ peak ratio using a measured intensity at 1760 nm (FIG. 6). FIG. 7 is a graph having a horizontal or x-axis corresponding to a measured CO₂ wt % and a vertical or y-axis corresponding to a determined CO₂ peak ratio using a measured intensity at 1800 nm. It is presently recognized from FIG. 4 that the peak ratios reduce or substantially remove the effect of temperature and pressure on the CO₂ peak(s).

However, these trends are not linear. For the relatively low and mid-range CO₂ training, the trend may be a represented by a model having a particular order. For example, the model may be quadratic model with second-order dependence. However, this model does not appear to be valid with the inclusion of the relatively high CO₂ concentration data. For the relatively high CO₂ concentration model, it is noted that a model of having a different order (e.g., fourth order model) to prescribe the relationship between the peak ratios for high CO₂ concentration data. The unknown parameters of fourth order model are obtained by the training procedure similar to before.

Accordingly, it is presently recognized that, to combine the low and mid-range CO₂ algorithm with the high CO₂ algorithm proposed in this memo, it may be advantageous to selectively utilize one of multiple models (e.g., a first model for the relatively low CO₂ concentration data and a second model for relatively high CO₂ concentration data) based on one or more ratios of the CO₂ measurement data. For example, as noted with respect to FIG. 2, the model used to generate the data depicted in the graph of FIG. 2 does not accurately determine CO₂ wt % for values above a CO₂ wt %. That is, it is presently recognized that above a weight percent threshold (e.g., relatively high CO₂ wt %), the model corresponding to relatively high CO be used. Accordingly, computing the peak ratio between a CO₂ peak and a hydrocarbon reference measurement (e.g., an optical density at a particular wavelength measurement data) and selecting a model to use to determine the CO₂ concentration based on a comparison the peak ratio with a threshold ratio, may provide a way to select which algorithm to use.

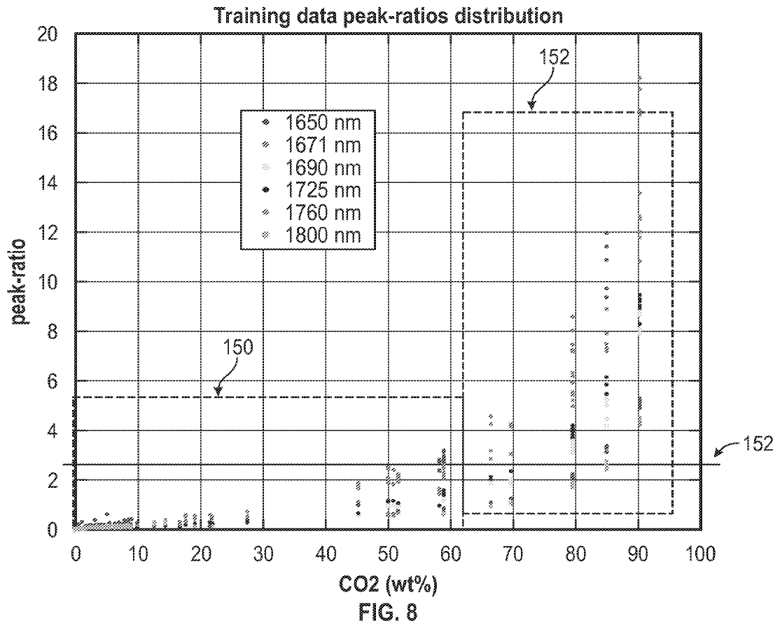

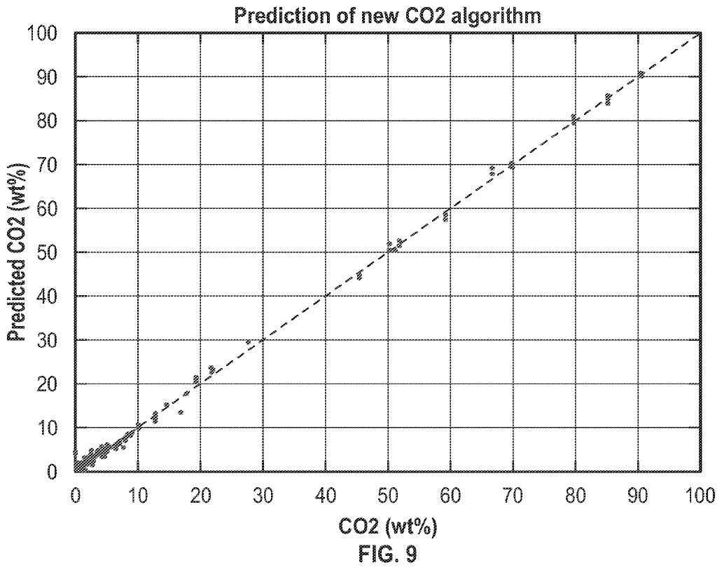

To illustrate this, FIG. 8 is a graph having a horizontal or x-axis corresponding to a measured CO₂ wt % and a vertical or y-axis corresponding to a determined CO₂ peak ratio normalized at different wavelengths. More specifically, the graph shows the distribution of computed peak ratios normalized with the reference channel data at six different wavelengths (e.g., 1650 nm, 1671 nm, 1690 nm, 1725 nm, 1760 nm, and 1800 nm) which are used to predict the CO₂ concentration. In general, the peak ratios within the region 190 fall under a certain peak threshold ratio, while the peak ratios within the region 192 are above the threshold ratio. Accordingly, the threshold value 194 may be used as a selective criteria for determine one of multiple models to use to determine a CO₂ wt % (e.g., CO₂ concentration) based on CO₂ measurement data. For example, if the processor 78 determines that at least one (e.g., a maximum of peak ratios normalized at six different wavelengths) is less than 2.5, the processor 78 may retrieve a first model corresponding to the low and mid-range CO₂ wt %. However, if the processor 78 determines that at least one (e.g., a maximum of peak ratios normalized at six different wavelengths) is greater than 2.5, the processor may retrieve a second model use corresponding to the high range CO₂ wt %. In either case, the processor 78 may utilize the retrieved model to determine the CO₂ concentration. The proposed strategy works well in the transition region in 50-60 wt %. For demonstration, FIG. 9 shows the prediction of CO₂ concentration using the spectral database and the second dataset and/or the third dataset as test data. With respect to the diagonal line, the predicted CO₂ values show a good agreement with the corresponding lab CO₂ (wt %) (i.e. ground truth) over the full range, which indicates that the fusing strategy of selectively utilizing one or more multiple models based on the peak ratios provides accurate measurements of the CO₂ concentration.

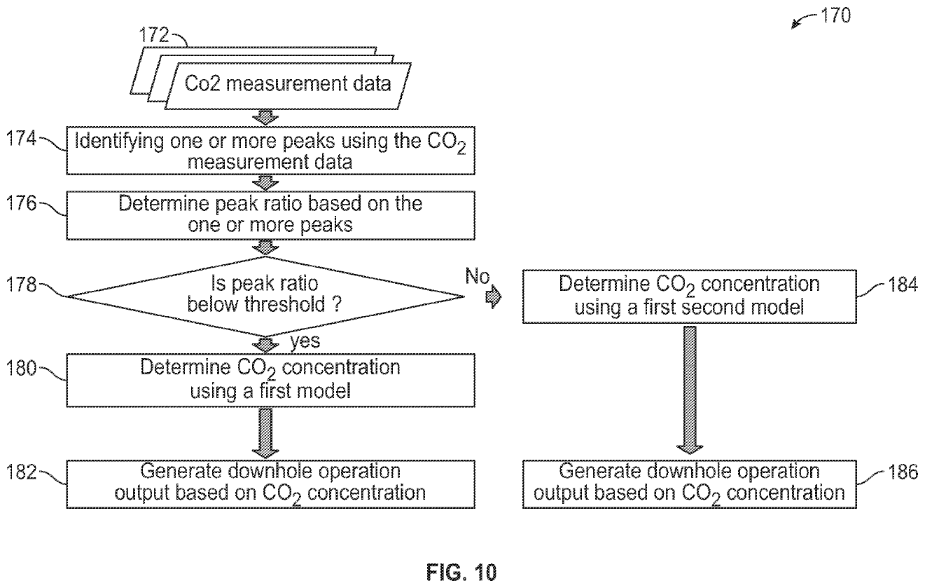

To generally illustrate this, FIG. 10 is a flowchart of a method 200 for selecting one or more multiple models based on calculated peak ratios, according to embodiments of the present disclosure. Any suitable device (e.g., a controller), such as the data processing system 76, may perform the method 200. In some embodiments, the method 200 may be implemented by executing instructions stored in a tangible, non-transitory, computer-readable medium, such as the memory 80 and/or storage 82, using the processor 78. For example, the method 200 may be performed at least in part by one or more software components, such as an operating system of the electronic device, such as a laptop, computer, or personal electronic device, one or more software applications of the electronic device, firmware of the electronic device, and the like. While the method 200 is described using steps in a specific sequence, it should be understood that the present disclosure contemplates that the described steps may be performed in different sequences than the sequence illustrated, and certain steps may be omitted.

The processor 78 receives CO₂ measurement data (i.e. optical spectra) 202 and identifies, at block 204, one or more peaks from the CO₂ measurement data. In general, the CO₂ measurement data may include a spectra over a range of wavelengths corresponding to certain transitions (e.g., vibrational transitions, rotational transitions, and so on) of CO₂. In some embodiments, the CO₂ measurement data may be acquired by a downhole CO₂ spectrometer. In general, the processor 78 may identify the peaks by comparing the intensity at each wavelength to a threshold, and if the intensity is above a threshold and/or distinct from noise, the processor 78 may identify the peaks, as should be appreciated by one of ordinary skill in the art.

At block 206, the processor 78 determines the peak ratio (e.g., the CO₂ peak ratio) using the one or more identified peaks. In general, the processor 78 may determine the peak ratio as described with respect to Eqns. 8 and 9. In some embodiments, the processor 78 may normalize the peak ratio to a hydrocarbon absorption peak in the hydrocarbon absorption region (i.e. 1600 nm-1800 nm), thereby removing certain dependencies as described herein.

At block 208, the processor 78 determines whether the computed peak ratios exceed a threshold ratio. In some embodiments, the threshold ratio may be a threshold ratio range, such as above 1.0, above 2.0, above, 2.5, above 3.0, and so on. In some embodiments, the threshold ratio may be a particular threshold value, such as 1.0, 2.0, 2.5, 3.0, 3.5, and so on. If the processor 78 determines that the peak ratio exceeds the threshold ratio, or is within the threshold range, then the processor 78 may proceed to block 210 and determine a CO₂ concentration using a first model. For example, the processor 78 may retrieve a first model stored in the memory 80. In general, the first model may be capable of producing an about CO₂ concentration using the CO₂ measurement data below an error threshold (e.g., 15%, 10%, 5%, 1%) for a first range of CO₂ concentrations (e.g., between 0 wt % to 50 wt %, between 5 wt % and 40 wt %, between 5 wt % and 30 wt %, and so). Accordingly, using the first model, the processor 78 may determine the CO₂ concentration and generate, at block 212, a downhole operation output based on the determined concentration at block 210 (e.g., using the first model corresponding to the error threshold for the first range of CO₂ concentrations). In general, the downhole operation output may be an alert, indicative of the determined CO₂ concentration, and/or a control signal that adjusts operation of a downhole tool. For example, the downhole operation output may cause the display 84 to depict a graphical user interface (GUI) indicating the determined CO₂ concentration. Additionally or alternatively, the downhole operation output may cause a component of the downhole tool 12 to activate or deactivate.

However, if at block 208, the processor 78 determines that the peak ratio is below the threshold ratio, or is outside the threshold range, then the processor 78 may proceed to block 214 and determine a CO₂ concentration using a second model. For example, the processor 78 may retrieve a second model stored in the memory 80. In general, the second model may be capable of outputting a CO₂ concentration using the CO₂ measurement data below an error threshold (e.g., 15%, 10%, 5%, 1%) for a second range of CO₂ concentrations (different than the first range of CO₂ concentrations) (e.g., between 50 wt % to 90 wt %, between 55 wt % and 95 wt %, between 40 wt % and 100 wt %, and so). Accordingly, using the second model, the processor 78 may determine the CO₂ concentration and generate, at block 216, a downhole operation output based on the determined concentration at block 214 (e.g., using the second model corresponding to the error threshold for the second range of CO₂ concentrations). In general, the downhole operation output may be generally similar to the downhole operation output described with respect to block 212.

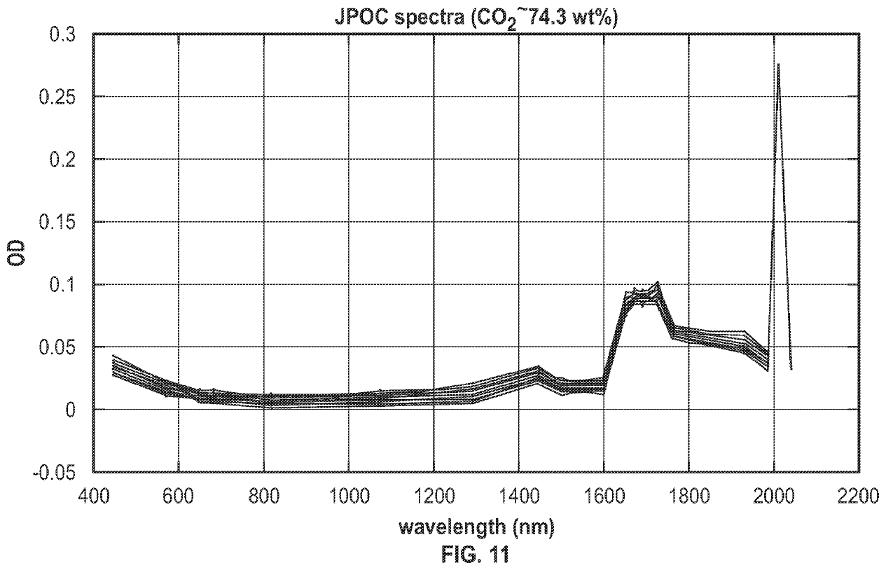

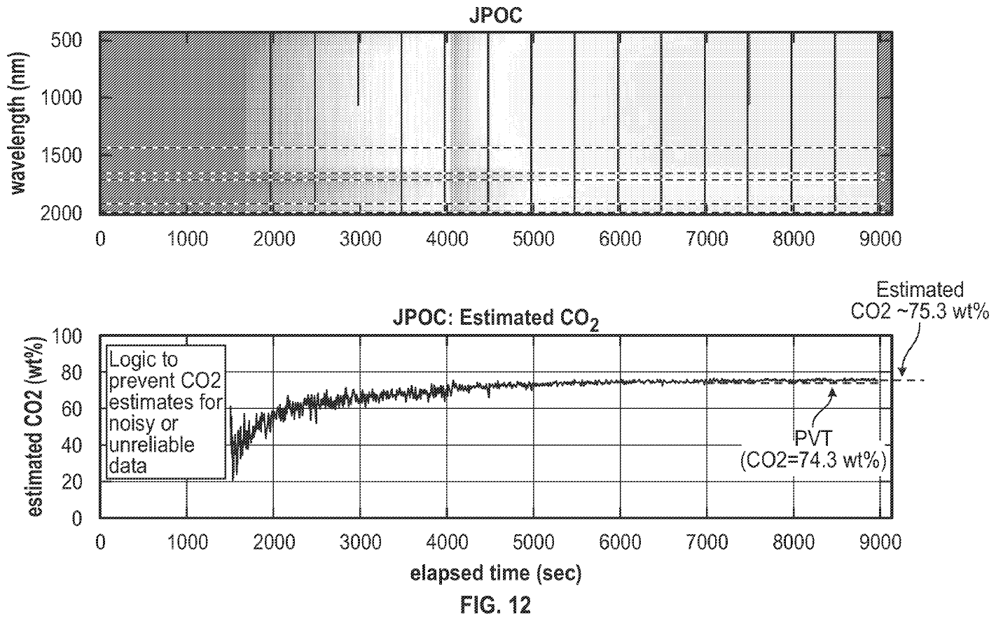

For validation of the disclose techniques, the disclosed techniques were applied to two field examples with the pressure-volume-temperature (PVT) reports of captured fluid samples available for comparison. The first example is spectral data from a first gas well. FIG. 11 shows the spectra from multiple snapshots. Other than some scattering offsets, all spectra appear to be consistent and show a large CO₂ absorption peak at 2010 nm. FIG. 12 shows a variable density log (VDL) of spectral data (top) and the estimated CO₂ wt % (bottom). Initially, the estimated CO₂ wt % is relatively low at about 20 wt % and with continuous pumping, the CO₂ concentration gradually increases and reaches 75.4 wt % at the end, which is nearly right on the PVT measured CO₂ concentration value (74.3 wt %) of the captured sample.

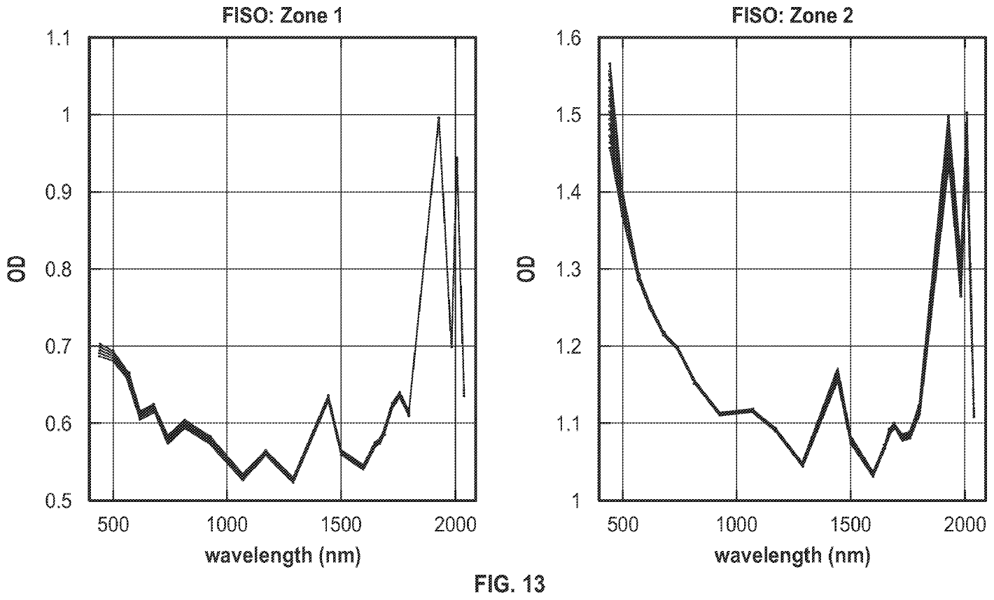

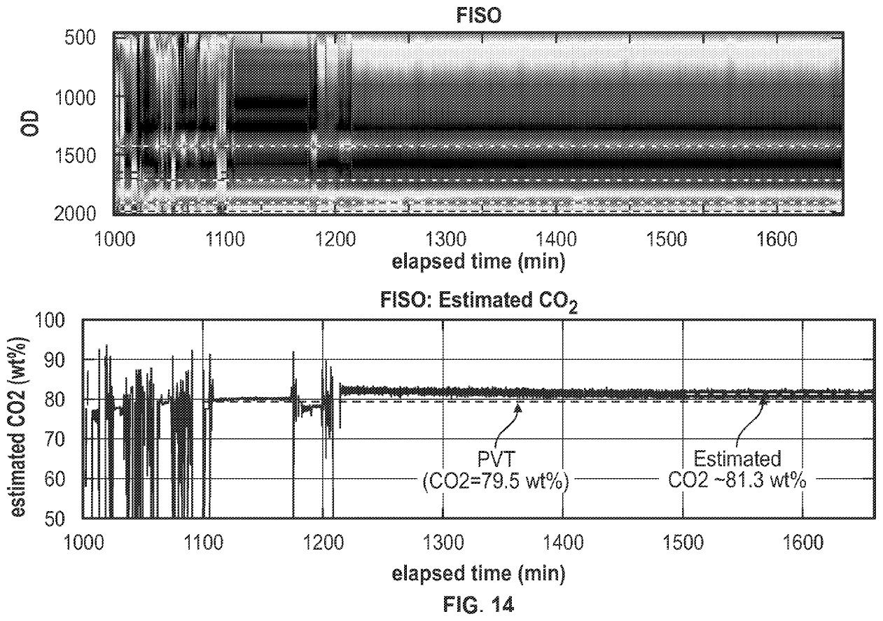

In the second example, the combined algorithm is applied to spectra data from a second gas well. Gas in the reservoir contain relatively high CO₂ concentrations according to the PVT reports. FIG. 13 shows the spectra from two different zones during pumping. Spectra in both zones show a strong CO₂ absorption peak at 2010 nm as well as a relatively strong water absorption peak at 1930 nm. The presence of water absorption is an interference to extracting the CO₂ peaks and peak ratios. FIG. 14 shows the VDL of spectra data (top) and the estimated CO₂ wt % (bottom). Even with some interference from the water absorption, the estimated CO₂ wt % in zone 1 is still quite close to the PVT measured CO₂ concentration value (79.6 wt %), whereas the estimated CO₂ wt % in zone 2 is slightly off (at about 81.3 wt %), which is still within the required accuracy of ±2 wt %.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A method, comprising:
receiving, via one or more processors, carbon dioxide (CO₂) optical spectrometer measurement data corresponding to a region within a geological formation;
identifying, via the one or processors, a peak corresponding to CO₂ based on the CO₂ measurement data;
determining, via the one or more processors, a ratio between the peak and a hydrocarbon reference measurement;
selecting, via the one or more processors, a model from a plurality of stored models for determining a CO₂ concentration based on the ratio;
determining, via the one or more processors, the CO₂ concentration using the selected model; and
generating, via the one or more processors, a downhole operation output based on the determined CO₂ concentration.

2. The method of claim 1, wherein the CO₂ measurement data is acquired by a downhole spectrometer.

3. The method of claim 1, wherein the plurality of models are trained based on reference spectral measurements using machine learning, artificial intelligence, or both.

4. The method of claim 1, wherein the hydrocarbon reference measurement comprises an optical density (OD) at a first wavelength within a wavelength range corresponding to the CO₂ measurement data.

5. The method of claim 1, wherein a first model of the plurality of models is configured to output the CO₂ concentration within a first range of concentrations, and wherein a second model of the plurality of models is configured to output the CO₂ concentration within a second range of concentrations different than the first range of concentrations.

6. The method of claim 5, wherein selecting, via the one or more processors, the model for determining the CO₂ concentration based on the ratio comprises:
determining that the ratio exceeds a threshold ratio; and
selecting the first model of the plurality of models based on the ratio exceeding the threshold ratio.

7. The method of claim 5, wherein selecting, via the one or more processors, the model for determining the CO₂ concentration based on the ratio comprises:
determining that the ratio is below a threshold ratio; and
selecting the second model of the plurality of models based on the ratio being below the threshold ratio.

8. A method, comprising:
receiving, via one or more processors, carbon dioxide (CO₂) optical spectrometer measurement data corresponding to a region within a geological formation;
identifying, via the one or processors, a peak corresponding to CO₂ based on the CO₂ measurement data;
determining, via the one or more processors, a plurality of ratios between the peak corresponding to CO₂ and a plurality of hydrocarbon peak measurements;
comparing, via the one or more processors, a ratio of the plurality of ratios to a threshold ratio;
selecting, via the one or more processors, a model from a plurality of stored models for determining a CO₂ concentration based on the comparison between the ratio and the threshold ratio;
determining, via the one or more processors, the CO₂ concentration using the selected model; and
generating, via the one or more processors, a downhole operation output based on the determined CO₂ concentration.

9. The method of claim 8, where the downhole operation output is configured to cause a graphical user interface to display the determined CO₂ concentration.

10. The method of claim 8, wherein determining, via the one or more processors, the ratio of the plurality of ratios is below the threshold ratio comprises:
determining a maximum ratio of the plurality of ratios; and
determining the maximum ratio of the plurality of ratios is below the threshold ratio.

11. The method of claim 8, wherein the plurality of stored models correspond to different ranges of CO₂ concentrations.

12. The method of claim 11, wherein the selected model of the plurality of stored models corresponds to a CO₂ concentration range greater than 50 weight percent (wt %).

13. The method of claim 8, wherein the plurality of hydrocarbon peak measurements comprise peaks corresponding to at least one of methane, ethane, propane, butane, pentane, or hexane.

14. The method of claim 8, wherein the downhole operation output is configured to adjust one or more operations of a downhole tool utilized within the geological formation.

15. The method of claim 8, wherein the threshold ratio is a number greater than 2.

16. A non-transitory computer-readable medium comprising computer-executable instructions that, when executed by a processor, are configured to cause the processor to:
receive carbon dioxide (CO₂) optical spectrometer measurement data corresponding to a region within a geological formation;
identify a peak corresponding to CO₂ based on the CO₂ measurement data;
determine a plurality of ratios between the peak corresponding to CO₂ and a plurality of hydrocarbon peak measurements;
compare a ratio of the plurality of ratios to a threshold ratio;
select a model from a plurality of stored models for determining a CO₂ concentration based on the comparison between the ratio and the threshold ratio;
determine the CO₂ concentration using the selected model; and
generate a downhole operation output based on the determined CO₂ concentration.

17. The non-transitory computer-readable medium of claim 16, wherein the instructions, when executed by the processor, cause the processor to:
determine that at least one ratio of the plurality of ratios is below an additional threshold ratio;
select an additional model for determining the CO₂ concentration based on the at least one ratio being below the threshold ratio; and
determine the CO₂ concentration using the selected additional model.

18. The non-transitory computer-readable medium of claim 16, where the downhole operation output is configured to cause a graphical user interface to display the determined CO₂ concentration.

19. The non-transitory computer-readable medium of claim 16, wherein the hydrocarbon peak measurement corresponds to an intensity of optical spectrometer measurement data at a wavelength corresponding to an alkane.

20. The non-transitory computer-readable medium of claim 16, wherein the threshold ratio is approximately 2.5.
